(19) United States
(12) Patent Application Publication
(10) Pub. No.: US 2011/0259355 A1
(43) Pub. Date: Oct. 27, 2011

(54) PERSONAL CARE PRODUCTS

(76) Inventors: Arnold Ybarra, League City, TX (US); Dalia Eicken, Houston, TX (US); Vinh Nguyen, Pearland, TX (US); Richard Vicari, Pearland, TX (US); Jeffrey Paul Coulson, Laegue City, TX (US); Philip Joel Brondsema, Houston, TX (US); Patricia Nunez De Arce, Lewisville, TX (US)

(21) Appl. No.: 13/059,086

(22) PCT Filed: Aug. 13, 2009
(86) PCT No.: PCT/US09/53704

§ 371 Date: May 2, 2011

(30) Foreign Application Priority Data
Aug. 15, 2008 (US) 61189073

(51) Int. Cl. A61K 8/81; A45D 7/04; A61Q 17/04; A61Q 5/06; A61Q 19/00

(52) U.S. Cl. 132/203; 424/70.16; 424/78.03; 424/59; 424/60

(57) ABSTRACT

A hair care product comprising water, a chelating agent, a copolymer, a pH adjusting agent and a hair treatment composition including a polyvinyl alcohol, polyvinyl alcohol copolymer, vinyl acetate emulsion, acrylic emulsion, vinyl acrylic emulsion, vinyl acetate copolymer emulsion or a combination thereof.

### RELATED CASES

This application claims the priority of the provisional application Ser. No. 61/189,073 filed Aug. 15, 2008.

### FIELD OF THE INVENTION

The invention relates to the use of polymers containing primary and secondary alcohols that may contain functional groups, in addition to the optional use of vinyl acetate emulsions, vinyl acrylic emulsions, acrylic and or vinyl acetate copolymer emulsions for personal care products.

### BACKGROUND OF THE INVENTION

An almost limitless variety of personal care products are commercially available to consumers. Hair care products, for instance, may be intended to enhance the manageability and look of one's hair. The hair care products are available in the form of creams, ointments, gels, foams, mousses, liquids, and semi-solid compositions. In addition to various functional ingredients, the hair care products can generally include pigments, fragrances, and various other additives.

In recent years, those skilled in the art have attempted to use different types of polymers in improving personal care products. Creating and selecting polymers, however, that are safe to use and that have a beneficial blend of properties has been somewhat problematic. In addition, many polymers do not perform well when combined with other components, while other polymers are not amenable to commercial scale production.

Polymers for use in hair care products such as mousses, gels and hair spray should possess various important properties and characteristics. For instance, the polymers should possess sufficient stiffness and humidity resistance to hold the hair in place under conditions normally encountered by the user. The polymers should also be readily removable from the hair by conventional shampoos and/or water. In this regard, a continuing need exists in the hair care market for a hair care product that possesses high humidity curl retention and provides sufficient stiffness to the hair without producing flakes. A need also exists for a material that will improve the shine/gloss, hold and manageability of hair when incorporated into a hair care product. A need also exists for a material, such as a polymer, that is also capable of enhancing or improving various properties of skin care products.

### SUMMARY OF THE INVENTION

A hair care product comprising water, a chelating agent, a copolymer, a pH adjusting agent and a hair treatment composition including a polyvinyl alcohol, polyvinyl alcohol copolymer, vinyl acetate emulsion, acrylic emulsion, vinyl acrylic emulsion, vinyl acetate copolymer emulsion or a combination thereof.

### DETAILED DESCRIPTION

The present invention relates to personal care compositions (i.e. hair care products, skin care products) containing one or more polymer additives. The polymer additive may comprise an N-vinyl formamide copolymer, a polyvinyl alcohol, a polyvinyl alcohol copolymer, a vinyl acetate emulsion, a vinyl acrylic emulsion, an acrylic, a vinyl acetate copolymer emulsions or a combination thereof. More specifically, the instant invention relates to a hair care product which is comprised of water, a chelating agent, a copolymer, a pH adjuster and a hair treatment composition. The hair treatment composition is comprised of a polymer containing primary and secondary alcohols that may contain functional groups (i.e., polyvinyl alcohol, polyvinyl alcohol copolymer), vinyl acetate emulsion, vinyl acrylic emulsion, acrylic, vinyl acetate copolymer emulsions, or a combination thereof. Of particular advantage, the copolymers of the instant invention may be modified and chemically altered in order to vary the properties of the resulting material. Thus, the polymer additives may be used in numerous different compositions for various purposes.

Hair care product, as used herein, refers to a product which may be used to treat or care for an individual's hair. The hair care product may be comprised of water, a chelating agent, a copolymer, a pH adjuster and a hair treatment composition. Hair care products may include, but are not limited to, hair sprays, hair gels, hair mousse, shampoos, conditioners, shower gels, bath products, cleansers, exfoliators, hair dyes, hair straighteners, hair curlers (i.e. permanents), waves, hair glossing products or combinations thereof. In one embodiment, the hair care product may be considered an “after shower” product which may be used after shampooing and/or conditioning one's hair.

Water, as used herein, refers to the chemical substance having the chemical formula of H₂O. In the instant invention, various types of water may be utilized which include, but are not limited to, mineral water, spring water, tap water, deionized water, distilled water, or a combination thereof. Water may be present in the instant invention in an amount generally between 75 and 99%, more preferably between 85 and 95%, and most preferably between 90 and 94%.

Chelating agent, as used herein, refers to a substance used to chelate or bind metallic ions, such as with a heterocyclic ring structure so that the ion is held by chemical bonds from each of the participating rings. Suitable chelating agents include ethylene diaminetetraacetic acid (EDTA), EDTA disodium, calcium disodium edetate, EDTA trisodium, EDTA tetrasodium and EDTA dipotassium. One or more chelating agents can optionally be included. Chelating agent may be present in the instant invention in an amount generally from between 0.01 and 1%, more preferably between 0.03 and 0.8%, and most preferably between 0.05 and 0.1% by weight.

Copolymer, as used herein, refers to a polymer derived from two or more monomers. Examples of copolymers include, but are not limited to, Ultrez 21, Cremophor RH40, Carbomer 940, Carbopol ETD 2020, or combinations thereof. Copolymer may be present in the instant invention in an amount generally from between 0.1 and 2%, more preferably between 0.3 and 1.5%, and most preferably between 0.5 and 1%.

The hair care product or skin care product of the instant invention can be formulated so as to have a pH in a desired range. For instance, the pH of the hair care product can generally be from about 4.0 to about 12.0, and particularly from about 4.5 to about 11. In order to adjust and control the pH of the care product, in one embodiment, the composition can contain a pH adjusting agent. pH adjusting agent, as used herein, refers to a material which may be utilized to either raise or lower the pH of another material or solution. pH adjusting agents may include, but are not limited to, sodium hydroxide, citric acid, succinic acid, triethanolamine, 2-amino-2-methyl-1-propanol, benzophenone 4, triethanolamines, or combinations thereof. pH adjusting agent may be present in the instant invention in an amount generally between 0.01 to 2%, more preferably between 0.02 and 1.5%, and most preferably between about 0.04 and 1.44%.

Hair treatment composition, as used herein refers to a composition which includes a polyvinyl alcohol, polyvinyl alcohol copolymer, vinyl acetate emulsion, acrylic emulsion, vinyl acrylic emulsion, vinyl acetate copolymer emulsion or a combination thereof. In another embodiment, a hair treatment composition may be comprised of a polyvinyl alcohol, a polyvinyl alcohol copolymer with a vinyl acetate, a vinyl acetate copolymer, an acrylic emulsion or a vinyl acrylic emulsion. In still another embodiment, the hair treatment composition is comprised of a polyvinyl alcohol copolymer with an emulsion. The emulsion may be selected from the group comprising: vinyl acrylic emulsion, a vinyl acetate emulsion, a vinyl acetate ethylene emulsion, an acrylic emulsion and, or combinations thereof.

In one embodiment of the instant invention, the hair treatment composition may further include a partially hydrolyzed polyvinyl alcohol, a fully hydrolyzed polyvinyl alcohol or a combination thereof.

In addition to hair care products, the present disclosure is also directed to various skin care products. Skin care products can include, but are not limited to, skin moisturizers, sunscreen compositions, eye and face makeup preparations, other topical cosmetic formulations, nail polishes, cleansers including shampoos and conditioners, shower gels and bath products. A common complaint that consumers make about skin care products, however, is that such products do not adhere well to the skin and have a tendency to wear off. In view of the above, a need also exists for a polymer that can improve the retention or other properties of skin care products.

In one embodiment, when incorporated into a skin care product, the polymer additive serves to improve adhesion between the composition and the skin. Thus, the polymer additive can be used in any composition that is intended to deliver a benefit to the skin. In particular embodiments, for instance, the polymer additive may be incorporated into skin moisturizers and sunscreen compositions.

Still other skin care products that may be formulated in accordance with the present disclosure include skin repair compositions, facial creams, any cream or lotions containing a medicament or skin active, acne creams, anti-aging compositions, eye and face makeup preparations including lipsticks, liquid and powder foundations, rouge, concealers, eye and lip liners, eyelash mascara, eye shadows, makeup pencils, and the like, relaxers, shaving creams, shaving lotions, nail polishes, indoor tanning compositions, and the like. In another embodiment of the instant invention, a skin care product may comprise a skin care product including a polyvinyl alcohol, polyvinyl alcohol copolymer, vinyl acetate emulsion, acrylic emulsion, vinyl acrylic emulsion, vinyl acetate copolymer emulsion, and, or combinations thereof. In still another embodiment, the skin care product may be a skin moisturizer or a sunscreen/sun protecting agent.

Skin treatment composition, as used herein refers to a composition which includes a polyvinyl alcohol, polyvinyl alcohol copolymer, vinyl acetate emulsion, acrylic emulsion, vinyl acrylic emulsion, vinyl acetate copolymer emulsion or a combination thereof. In another embodiment, a skin treatment composition may be comprised of a polyvinyl alcohol, a polyvinyl alcohol copolymer with a vinyl acetate, a vinyl acetate copolymer, an acrylic emulsion or a vinyl acrylic emulsion. In still another embodiment, a skin treatment composition is comprised of a polyvinyl alcohol copolymer with an emulsion. The emulsion may be selected from the group comprising: vinyl acrylic emulsion, a vinyl acetate emulsion, a vinyl acetate ethylene emulsion, an acrylic emulsion and, or combinations thereof.

In accordance with the present disclosure, the skin treatment product may contain a polyvinyl alcohol copolymer. The polyvinyl alcohol copolymer has been found to improve adhesion of the composition to the skin or one or more other properties.

For example, when formulated into a sunscreen lotion, the composition may contain a sunscreen agent. Examples of sunscreen agents that may be used include zinc oxide, titanium dioxide, iron oxide, a para-amino benzoate, a salicylate, a cinnamate, a benzophenone, a menthyl anthralinate, a digalloyl trilleate, or mixtures thereof.

When formulated as a skin moisturizer, any suitable components and ingredients may be contained in the composition. A non-exhaustive and exemplary list of ingredients that may be contained in the skin moisturizer include, for instance, glycerine, a dimethicone or a derivative thereof, propylene glycol, lanolin or a derivative thereof, petrolatum, or mixtures thereof.

In one embodiment of the instant invention, a skin treatment composition may further include a partially hydrolyzed polyvinyl alcohol, a fully hydrolyzed polyvinyl alcohol or a combination thereof.

The polymer mentioned above may have the following molecular structure:

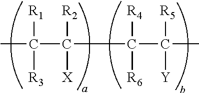

wherein: R1-R6 being selected from the group comprising: hydrogen, C₁-C₄ alkyl, C₁-C₄ alkoxy, acetate, hydroxyl, carbocyclic, heterocyclic or combinations thereof); and
X and Y being selected from the group comprising: hydroxyl, acetate, amine, amide, sulfonate, carboxylate, heterocyclic or combinations thereof.

For the instant invention, any known process may be utilized in order to synthesize the polyvinyl alcohol or polyvinyl alcohol copolymers. For example, the polyvinyl alcohol copolymers may be formed through processes which include, but are not limited to, free radical polymerization, grafting or redox initiation. Polyvinyl alcohol copolymers are described in U.S. Pat. No. 5,300,566 and U.S. Pat. No. 5,632,977, both of which are incorporated herein by reference.

In one embodiment, polyvinyl alcohol is manufactured from vinyl acetate monomer via a multi-step process. Vinyl acetate monomer is polymerized into polyvinyl acetate and then converted to polyvinyl alcohol. Polyvinyl alcohol is particularly advantageous to colorfastness because it is a safe, biodegradable, water soluble polymer. The above polyvinyl alcohol may have a molecular weight in the range of 1,000 to 1,000,000, more preferably in the range of 20,000 to 150,000. The above polyvinyl alcohol may be used in a concentration ranging from 0.1 to 50 grams per liter (g/L) when mixed with water.

In another embodiment, a copolymer of polyvinyl alcohol and N-vinyl formamide is formed by polymerizing a vinyl acetate monomer and an N-vinyl formamide monomer and then saponifying a copolymer of vinyl acetate and N-vinyl formamide. For example, a copolymer of vinyl acetate and N-vinyl formamide may be formed by combining the monomers within a reactor in the presence of a free radical initiator and an alcohol, such as methanol. The resulting reaction yields an intermediate vinyl acetate random copolymer containing vinyl formamide groups in an amount from about 1 mole percent to about 50 mole percent.

The resulting vinyl acetate and N-vinyl formamide copolymer is then subjected to saponification. The copolymer is saponified by subjecting the copolymer to heat in the presence of an alkali material. The alkali material may be selected from the group which includes, but is not limited to, any suitable base such as sodium hydroxide, potassium hydroxide, or an alkali metal alkolate. Saponification can be carried out in a continuous system, a semi-batch system, or in a batch system. In one embodiment, an alkali may be added to one of the above described copolymers in a caustic mole ratio (CMR, ratio of base to acetate groups on the polymer) in an amount from about 0.01% to about 0.1%. The copolymer is then heated to a temperature of from about 20° C. to about 50° C. and for a period of time of from about 5 minutes to about 24 hours. During saponification, the vinyl acetate is converted into a vinyl alcohol. The degree of saponification may be controlled and adjusted by controlling the saponification conditions. However, it is common that 95% or more of the available vinyl acetate units are converted into vinyl alcohol.

After saponification, the resulting product can be subjected to various washing steps in order to remove any acetates or other impurities. The resulting polyvinyl alcohol and N-vinyl formamide copolymer can have the following general formula:

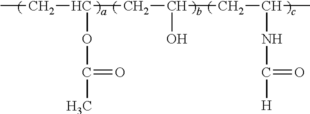

where:
a is from about 0 to about 15 mole %,
b is from about 0.1 mole % to about 99 mole %, and
c is from about 0.1 mole % to about 99 mole %.
   The above copolymer may have a molecular weight in the range of 1,000 to 1,000,000, more preferably in the range of 20,000 to 250,000.

As shown above, the copolymer can be formulated to only contain free hydroxyl groups and free amide groups. Copolymers made according to the above formula may be used in accordance with the instant invention and have been found to provide specific advantages in some embodiments. For example, the above described copolymer is well suited for blending with emulsifiers.

In another embodiment, the above polyvinyl alcohol and N-vinyl formamide copolymer can be further hydrolyzed by either an acid or a base in order to incorporate amine groups into the copolymer. The hydrolysis of the polyvinyl alcohol and N-vinyl formamide copolymer may be conducted in a continuous system, a semi-batch system, or in a batch system in the presence of an acid or base to serve as a catalyst. Hydrolysis can be conducted on the copolymer in a slurry in the presence of a solvent. The solvent may comprise an alcohol, such as methanol. Of particular advantage, hydrolysis can be controlled in order to produce a copolymer having properties tailored to a particular application. Thus, the hydrolysis can be either substantially complete or partial.

When hydrolysis of the copolymer occurs in the presence of a base, the base may be selected from a group which includes, but is not limited to, an alkali hydroxide or an alkaline earth hydroxide. In one embodiment, hydrolysis is carried out in the presence of either sodium hydroxide or potassium hydroxide. The base is present in an amount from about 0.5 to about 3 times the stoichiometric quantity of N-vinyl formamide groups present. Hydrolysis can also be carried out at elevated temperatures, such as from about 50 to about 80° C. The resulting product can be recovered by filtration or solvent evaporation. In another embodiment, the base is present in an amount from about 1 to about 1.5 times the stoichiometric quantity of N-vinyl formamide groups present.

Hydrolysis of the polyvinyl alcohol and N-vinyl formamide copolymer may also occur in the presence of various acids which include, but are not limited to, halogen acids, citric acid, succinic acid, trifluoroacetic acid and methane sulfonic acid. In one embodiment, hydrolysis is carried out in the presence of hydrochloric acid. Acid hydrolysis may be conducted at a temperature of from about 15° C. to about 80° C. The acid may be present in an amount from about 0.5 to about 3 times the stoichiometric amount of N-vinyl formamide groups present. In another embodiment, the acid may be present in an amount from about 1 to about 1.5 times the stoichiometric amount of N-vinyl formamide groups present. Acid hydrolysis may also be conducted in a methanol suspension.

After hydrolysis, a copolymer having the following formula can be formed:

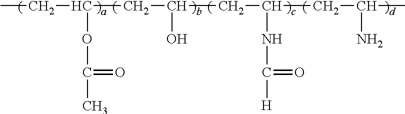

### Where:

a is from about 0 mole % to about 15 mole %;
b is from about 0.1 mole % to about 99 mole %;
c is from about 0 mole % to about 30 mole %; and
d is from about 0.1 mole % to about 50 mole %.
   The above copolymer may have a molecular weight in the range of 1,000 to 1,000,000, more preferably in the range of 20,000 to 250,000.

Of particular advantage, hydrolysis can be controlled in order to produce a copolymer with properties tailored to a particular application. Thus, the hydrolysis can be substantially complete or it can be partial.

Once produced, the polyvinyl alcohol and N-vinyl formamide copolymer can be incorporated into numerous hair treatment compositions. When formulating a hair treatment composition containing the copolymer, the copolymer can be combined with numerous different ingredients and additives depending upon the particular application and the desired result. A non-exhaustive list of additives that may be combined with the N-vinyl formamide copolymer may include, but is not limited to, rheology modifiers, thickeners, surfactants, pigments, dispersions aids, moisture retention agents, waxes, oils, pigment, encapsulating agents, vitamins, active ingredients, proteins, preservatives, plasticizers, film formers, hydrating substances, silicones, UV filters and absorbers, alpha-hydroxyacids, antioxidants, essential oils, emulsifiers and emulsion stabilizers, emulsifying agents, sensory modifiers, humectants, slip agents, solvency, gelling agents, fragrance fixatives, foam enhancers, deposition agents, hair protectants, opacifiers, pearlizers, plant extracts, polyquaterniums or combinations thereof.

A polyvinyl alcohol and N-vinyl formamide copolymer as described above can also be blended with one or more stability or performance additives in order to further enhance one or more properties of the resulting composition. The stability or performance additive may comprise, for instance, a polyvinyl pyrrolidone, a poly(vinyl acetate-co-N-vinyl-2-pyrrolidone) copolymer, vinyl caprolactum, a polyvinyl fluoride polymer, a styrene maleic anhydride copolymer, an acrylic polymer, a hydrophobically modified polyvinyl alcohol, a quaternary amine, a polyquaternium, an amine scavenging agent, a cationic surfactant, an amphoteric surfactant, or mixtures thereof.

The particular N-vinyl formamide copolymer and the one or more additives combined with the copolymer can be selected in order to enhance various properties of the resulting composition depending upon the particular end use application. The stability or performance additive, for instance, may increase the storage stability of the personal care composition, may decrease haze or the formation of haze, or may be used to control the viscosity, rheology and/or the pH of the personal care composition. When producing hair treatment compositions in accordance with the present disclosure, such as hair styling or hair fixative compositions, the polymer blend can improve the appearance and curl memory of hair without producing any substantial associate flaking. Hair treated with compositions according to the present disclosure, for instance, provides desirable amounts of stiffness to the hair and may also improve shine/gloss. Ultimately, the hold and manageability of the hair can be dramatically improved.

In one embodiment an N-vinyl formamide copolymer as described above may be blended with a polyvinyl pyrrolidone copolymer. Polyvinyl pyrrolidone copolymers are water soluble film-forming polymers that are particularly well adapted to absorbing water vapor. Thus, polyvinyl pyrrolidone copolymers may further improve curl retention in hair care products when combined with N-vinyl formamide copolymers, especially in high humidity environments. Exemplary polyvinyl pyrrolidone copolymers are available under the trade name OmniPur® from EMD Chemicals Inc. When polyvinyl pyrrolidone copolymers are included in a hair or skin care product, the polyvinyl pyrrolidone copolymer is present in an amount of between about 0.5 to about 10.0 by weight of the total composition, preferably between about 1.0 to about 6.0, and more preferably between about 2.0 to about 4.0.

In another embodiment, an N-vinyl formamide copolymer can be combined with a poly(vinyl acetate-co-N-vinyl-2-pyrrolidone) copolymer. The poly(vinyl acetate-co-N-vinyl-2-pyrrolidone) copolymer is formed from vinyl acetate monomers and N-vinyl-2-pyrrolidone monomers, where the vinyl acetate monomer is present in the copolymer in an amount between about 30 to 70% by weight of the copolymer. The poly(vinyl acetate-co-N-vinyl-2-pyrrolidone) copolymer may serve to improve curl retention, especially in high humidity environments. Poly(vinyl acetate-co-N-vinyl-2-pyrrolidone) copolymers are film-forming agents. The copolymers are marketed under the name “PVP/VA” by the company (ISP, BASF) in the form a powder for the “PVP/VA-S” series, or in a solution in ethanol or isopropanol respectively for the series “PVP/VA-E” and “PVP/VA-1” or by the company BASF under the name KOLLIDON VA. When a poly(vinyl acetate-co-N-vinyl-2-pyrrolidone) copolymer is included in a hair or skin care product, the poly(vinyl acetate-co-N-vinyl-2-pyrrolidone) copolymer is present in an amount of between about 0.5 to about 10.0 by weight of the total composition, preferably between about 0.5 to about 6.0, and more preferably between about 1.0 to about 4.0.

In another embodiment, an N-vinyl formamide copolymer can be combined with vinyl caprolactum. Vinyl caprolactum is a nonionic water soluble polymer and may serve to improve the hydrophobic nature of the resulting composition or product. When combined with an N-vinyl formamide copolymer and contained in a hair treatment product, vinyl caprolactum polymers may serve to further improve curl retention when applied to the hair. When vinyl caprolactum polymers are included in a hair or skin care product, the vinyl caprolactum polymer is present in an amount of between about 0.1 to about 5.0 by weight of the total composition, preferably between about 0.1 to about 3.0, and more preferably between about 0.1 to about 2.0.

In another embodiment, an N-vinyl formamide copolymer can be combined with a polyvinyl fluoride polymer. Similar to vinyl caprolactum, polyvinyl fluoride polymers are hydrophobic agents. Thus, when combined with an N-vinyl formamide copolymer, the polyvinyl fluoride polymer may serve to make the resulting composition more resistant to humidity and may increase curl retention when contained in a hair care product. When contained in a skin care product, on the other hand, the polyvinyl fluoride polymer may further improve skin adhesion and water resistance. When polyvinyl fluoride polymers are included in a hair or skin care product, the polyvinyl fluoride polymer is present in an amount of between about 0.1 to about 5.0 by weight of the total composition, preferably between about 0.1 to about 3.0, and more preferably between about 0.1 to about 2.0.

In another embodiment, an N-vinyl formamide copolymer can be combined with a styrene maleic anhydride copolymers. Styrene maleic anhydride copolymers are synthetic copolymers that are constructed out of styrene and maleic anhydride monomers. Styrene maleic anhydride copolymers are hydrophobic agents. Thus, the addition of styrene maleic anhydride copolymers may serve to improve the hydrophobic nature of the composition or product and provide improved humidity resistance. Furthermore, the styrene maleic anhydride copolymer may serve to lower the haze of the composition. Still further, the styrene maleic anhydride copolymer may also serve to improve the storage life of the composition. Suitable styrene maleic anhydride copolymers are available under the trade name Xiran® from Polyscope Polymers, the trade name SMA® from Sartomer, and the trade name Dylark® from Nova Chemicals. When styrene maleic anhydride copolymers are included in a hair or skin care product, the styrene maleic anhydride copolymer is present in an amount of between about 0.1 to about 5.0 by weight of the total composition, preferably between about 0.1 to about 3.0, and more preferably between about 0.5 to about 2.5.

In another embodiment, an N-vinyl formamide copolymer can be combined with a hydrophobically modified polyvinyl alcohol. The hydrophobically modified polyvinyl alcohol may serve to improve the products resistance to water and improve curl retention, especially in high humidity environments. When contained in a skin care product, on the other hand, the hydrophobically modified polyvinyl alcohol may serve to further improve skin adhesion and water resistance. When hydrophobically modified polyvinyl alcohol polymers are included in a hair or skin care product, the hydrophobically modified polyvinyl alcohol polymer is present in an amount of between about 0.5 to about 5.0 by weight of the total composition, preferably between about 0.5 to about 3.0, and more preferably between about 0.5 to about 2.0.

In another embodiment, an N-vinyl formamide copolymer can be combined with quaternary ammonium salts or quaternary amines. Quaternary amines are salts of quaternary ammonium cations with an anion. The quaternary amine may serve to improve the storage life or the haze of the composition or product. A suitable quaternary amine is dialklamidoethyl hydroxyethylmonium methosulfate. When quaternary amines are included in a hair or skin care product of the present invention, the quaternary amine is present in an amount of between about 0.1 to about 5.0 by weight of the total composition, preferably between about 0.5 to about 3.0, and more preferably between about 0.5 to about 2.0.

In another embodiment, an N-vinyl formamide copolymer can be combined with a polyquaternium polymer. A polyquaternium is a polymer class defined by having quaternary ammonium functional groups. Polyquaternium polymers may serve to improve the storage life or the haze of the composition. Suitable polyquaternium polymers may include: polyquaternium 3, polyquaternium 4, polyquaternium 10, polyquaternium 24, polyquaternium 2, polyquaternium 6, polyquaternium 7, polyquaternium 11, polyquaternium 16, polyquaternium 17, polyquaternium 18, polyquaternium 22, polyquaternium 24, polyquaternium 27, polyquaternium 28, polyquaternium 31, polyquaternium 32, polyquaternium 39, polyquaternium 41, polyquaternium 42, and quaternium 80. A preferred polyquaternium polymer is polyquaternium-24 available under the name Quatrisoft® polymer LM-200, from Amerchol Corporation. When a polyquaternium polymer is included in a hair or skin care product of the present invention, the polyquaternium polymer is present in an amount of between about 0.1 to about 10 by weight of the total composition, preferably between about 0.1 to about 5.0, and more preferably between about 0.5 to about 3.0.

In another embodiment, an N-vinyl formamide copolymer can be combined with an amine scavenging additive such as guanidine. The amine scavenging additive may provide improved high temperature storage stability, especially in environments over 45° C. Essentially, when other ingredients break down at elevated temperatures, the amine scavenging additive binds up and reacts with free amines and/or ammonia to keep them from reacting with and breaking down the N-vinyl formamide copolymer. Other suitable amine scavenging additives may include carboxylic anhydrides and copolymers of carboxylic anhydrides, aromatic anhydrides, isocyanates, polyisocyanates, and epoxides. When present, the amine scavenging additive can be present in an amount generally from about 0.05% to about 1.0% by weight of the hair treatment or skin treatment composition.

The polyvinyl alcohol copolymer emulsions described above utilize the emulsions described below. Emulsion, as used herein, refers to a mixture of two or more immiscible liquids held in suspension by small percentages of substances called emulsifiers. Emulsions may be either a natural or a synthetic compound. Natural compounds include proteins, carbohydrate polymers and/or fatty acids. Synthetic compounds include long-chain alcohols, acrylics, vinyl acrylics, vinyl acetates, and vinyl acetate ethylenes. Emulsions may include, but are not limited to, surfactant stabilized vinyl acrylic emulsions, vinyl acetate emulsions, vinyl acetate ethylene emulsions, or combinations thereof. In the instant invention, the concentration of the emulsion used may be in the range of 0.5 g/L to 100.0 g/L.

Thickener, as used herein, refers to a chemical substance which, when added to a mixture, increase its viscosity without substantially modifying its other properties. A thickener may be combined with a polyvinyl alcohol, a polyvinyl alcohol copolymer, a vinyl acetate emulsion, a vinyl acrylic emulsion, a vinyl acetate copolymer emulsion, or combinations thereof. Thickeners that may be used include, for instance, various acrylate polymers sold under the trade name CARBOPOL by the (Noveon, Lubrisol). Thickeners may include, but are not limited to, homopolymers of acrylic acid, homopolymers of acrylic acid crosslinked with an allyl ether pentaerythritol, allyl ether of sucrose, or allyl ether of propylene. In one embodiment, a C10 to C30 acrylate crosspolymer may be used as the thickener. In one embodiment of the instant invention, a thickener may be used in conjunction with a biocide, a preservative, a surfactant, or a combination thereof. In the instant invention, the amount of thickener used may be in the range of between 0.05 and 1%, preferably between 0.1 and 0.8%; and most preferably between 0.15 and 0.3% by weight.

Preservative, as used herein, refers to a chemical additive that aids in extending the shelf life of the hair care product. A preservative may be combined with a polyvinyl alcohol, a polyvinyl alcohol copolymer, a vinyl acetate emulsion, a vinyl acrylic emulsion, a vinyl acetate copolymer emulsion, or combinations thereof. Preservatives include, but are not limited to, citric acid, tartaric acid, phosphoric acid, iminodiacetic acid, nitrilotriacetic acid, hydroxyethyleneaminodiacetic acid and ethylenediaminetetraacetic acid and salts thereof; para-hydroxybenzoates such as butyl paraben, methyl paraben and propyl paraben; imidazolines (e.g., imidiazolinylurea), triclosan, hydantoins (e.g., dimethyloldimethylhydantoin), isothiazolidinone compounds and mixtures thereof. Commercially available preservatives include KATHON CG and KATHON CGII, which contain methylchloroisothiazolinone and methylisothiazolinone (Rohm and Haas). Preservatives can be present in an amount generally less than about 5% by weight, such as from about 0.01% to about 2% by weight. In one embodiment of the instant invention, a preservative may be used in conjunction with a biocide, a thickener, a surfactant, or a combination thereof.

Biocide, as used herein, refers to a chemical substance which is capable of killing one or more living organisms. A biocide may be utilized to protect a hair care product against biological infestation and growth. One or more biocides may be combined with a polyvinyl alcohol, a polyvinyl alcohol copolymer, a vinyl acetate emulsion, a vinyl acrylic emulsion, an acrylic emulsion, a vinyl acetate copolymer emulsion, or combinations thereof. Biocides may include, but are not limited to, oxidizing agents (e.g. chlorine, chlorine dioxide, chloroisocyanurates, hypochlorite, ozone), non-oxidizing agents (acrolein, amines, chlorinated phenolics, copper salts, organo-sulphur compounds, quaternary ammonium salts) or combinations thereof. In the instant invention, the amount of biocide used may be in the range of between 0.1 and 5%, preferably between 0.3 and 1.5%; and most preferably between 0.5 and 1% by weight. In one embodiment of the instant invention, a biocide may be used in conjunction with a preservative, a thickener, a surfactant, or a combination thereof.

Surfactant, as used herein, refers to a chemical additive that is a wetting agent that lowers the surface tension of a liquid, allowing easier spreading, and lowering the interfacial tension between two liquids. A surfactant may be combined with a polyvinyl alcohol, a polyvinyl alcohol copolymer, a vinyl acetate emulsion, a vinyl acrylic emulsion, a vinyl acetate copolymer emulsion, or combinations thereof. The N-vinyl formamide copolymer of the present disclosure may be combined with one or more surfactants. Surfactants include, but are not limited to, amphoteric surfactants and cationic surfactants. In the instant invention, the surfactant used may be in the range of between 0.5 and 10%, preferably between 1 and 6%; and most preferably between 3 and 5% by weight.

The term “amphoteric surfactant,” as used herein, is also intended to encompass zwitterionic surfactants, which are well known to formulators skilled in the art as a subset of amphoteric surfactants. A wide variety of amphoteric lathering surfactants can be used in the compositions of the instant invention. Particularly useful are those which are broadly described as derivatives of aliphatic secondary and tertiary amines, preferably wherein the nitrogen is in a cationic state, in which the aliphatic radicals can be straight or branched chain and wherein one of the radicals contains an ionizable water solubilizing group, e.g., carboxy, sulfonate, sulfate, phosphate, or phosphonate. Examples of amphoteric or zwitterionic surfactants include, but are not limited to, those selected from the group consisting of betaines, sultaines, hydroxysultaines, alkyliminoacetates, iminodialkanoates, aminoalkanoates or combinations thereof. Examples of betaines include, but are not limited to, the higher alkyl betaines, such as coco dimethyl carboxymethyl betaine, lauryl dimethyl carboxymethyl betaine, lauryl dimethyl alphacarboxyethyl betaine, cetyl dimethyl carboxymethyl betaine, cetyl dimethyl betaine (available as Lonzaine 16SP from Lonza Corp.), lauryl bis-(2-hydroxyethyl) carboxymethyl betaine, oleyl dimethyl gamma-carboxypropyl betaine, lauryl bis-(2-hydroxypropyl)alpha-carboxyethyl betaine, coco dimethyl sulfopropyl betaine, lauryl dimethyl sulfoethyl betaine, lauryl bis-(2-hydroxyethyl)sulfopropyl betaine, amidobetaines and amidosulfobetaines (wherein the RCONH(CH₂)₃ radical is attached to the nitrogen atom of the betaine), oleyl betaine (available as amphoteric Velvetex OLB-50 from Henkel), and cocamidopropyl betaine (available as Velvetex BK-35 and BA-35 from Henkel). Examples of sultaines and hydroxysultaines include, but are not limited to, materials such as cocamidopropyl hydroxysultaine (available as Mirataine CBS from Rhone-Poulenc).

Further examples of amphoteric surfactants of the instant invention include the following compounds: Cetyl dimethyl betaine (this material also has the CTFA designation cetyl betaine); Cocamidopropylbetaine; Cocamidopropyl hydroxy sultaine. Examples of other useful amphoteric surfactants are alkyliminoacetates, and iminodialkanoates and aminoalkanoates of the formulas RN[(CH₂)CO₂M]₂ and RNH(CH₂).mCO₂M wherein m is from 1 to 4, R is a C₈-C₂₂ alkyl or alkenyl, and M is H, alkali metal, alkaline earth metal ammonium, or alkanolammonium. Also included are imidazolinium and ammonium derivatives. Specific examples of suitable amphoteric surfactants include sodium 3-dodecyl-aminopropionate, sodium 3-dodecylaminopropane sulfonate, and N-higher alkyl aspartic acids. Other examples of useful amphoterics include amphoteric phosphates, such as coamidopropyl PG-dimonium chloride phosphate (commercially available as Monaquat PTC, from Mona Corp.). Also useful are amphoacetates such as disodium lauroamphodiacetate, sodium lauroamphoacetate, and mixtures thereof.

In another embodiment, the N-vinyl formamide copolymer can be combined with at least one cationic surfactant. Suitable cationic surfactants include, but are not limited to, fatty amines, di-fatty quaternary amines, tri-fatty quaternary amines, imidazolinium quaternary amines, and combinations thereof. Suitable fatty amines include monalkyl quaternary amines such as cetyltrimethylammonium bromide. A suitable quaternary amine is dialklamidoethyl hydroxyethylmonium methosulfate. Additional examples of cationic surfactant include: stearyldimenthylbenzyl ammonium chloride; dodecyltrimethylammonium chloride; nonylbenzylethyldimethyl ammonium nitrate; tetradecylpyridinium bromide; laurylpyridinium chloride; cetylpyridinium chloride; laurylpyridinium chloride; laurylisoquinolium bromide; ditallow(Hydrogenated)dimethyl ammonium chloride; dilauryldimethyl ammonium chloride; and stearalkonium chloride.

The total surfactants may be present in the hair care products in an amount of about 0.1 to about 20%, or about 0.1 to about 10%, or about 0.1 to about 5%. In one embodiment of the instant invention, a surfactant may be used in conjunction with a preservative, a thickener, a biocide, or a combination thereof.

The instant invention may be realized in multiple different embodiments. One particular embodiment of the instant invention may comprise a hair care product comprising water, a chelating agent, a copolymer, one or more pH adjusting agents, and a hair treatment composition including a polyvinyl alcohol copolymer. The polyvinyl alcohol copolymer of the hair treatment composition is comprised of a polyvinyl alcohol and an N-vinyl formamide copolymer having a molecular weight in the range of 10,000 to 250,000 and having the following molecular structure:

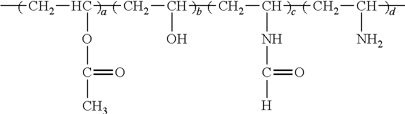

Where: a=0; b=90-98; c=0.1-15; and d=0-15.

Another embodiment of the instant invention may comprise a hair care product comprising water, a chelating agent, a copolymer, one or more pH adjusting agents, a preservative, and a hair treatment composition including a polyvinyl alcohol copolymer. The polyvinyl alcohol copolymer of the hair treatment composition is comprised of a polyvinyl alcohol and an N-vinyl formamide copolymer having a molecular weight in the range of 10,000 to 250,000 and having the following molecular structure:

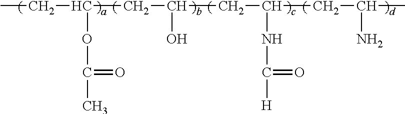

Where: a=0; b=90-98; c=0.1-15; and d=0-15.

Another embodiment of the instant invention may comprise a hair care product comprising water, a chelating agent, a copolymer, a pH adjusting agent, and a hair treatment composition including a polyvinyl alcohol copolymer. The polyvinyl alcohol of the colorfast composition has a molecular weight in the range of 10,000 to 250,000 and having the following molecular structure:

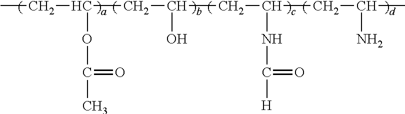

Where a=0-13; b=87-99.9; c=0; and d=0. The vinyl acetate copolymer emulsion is a surfactant stabilized emulsion.

Still another embodiment of the instant invention may comprise a hair care product comprising water, a chelating agent, a copolymer, one or more pH adjusting agents, a preservative, and a hair care treatment composition including a polyvinyl alcohol copolymer.

Yet another embodiment of the instant invention may comprise a hair care product comprising water, a chelating agent, a copolymer, one or more pH adjusting agents, a biocide, a hydrolyzed polyvinyl alcohol, and a hair care treatment composition including a polyvinyl alcohol copolymer.

Still another embodiment of the instant invention may comprise a hair care product comprising water, a chelating agent, a copolymer, one or more pH adjusting agents, a biocide, a partially hydrolyzed polyvinyl alcohol, and a hair care treatment composition including a polyvinyl alcohol copolymer.

Yet another embodiment of the instant invention may comprise a hair care product comprising water, a chelating agent, a copolymer, one or more pH adjusting agents, a thickener, and a hair care treatment composition including a polyvinyl alcohol copolymer.

The instant invention also includes a method for improving the curl retention of hair which comprises the steps of providing a hair care product comprising water, a chelating agent, a copolymer, a pH adjuster, and a hair treatment composition including a polyvinyl alcohol, a polyvinyl alcohol copolymer, a vinyl acetate emulsion, an acrylic emulsion, a vinyl acrylic emulsion, a vinyl acetate copolymer emulsion, or combinations thereof; and applying said hair care product to a portion or all of an individual's hair; and curling a portion or all of said individual's hair.

The polymer described in the method above may have the following molecular structure:

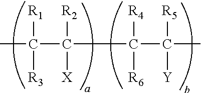

wherein: R1-R6 being selected from the group comprising: hydrogen, C₁-C₄ alkyl, C₁-C₄ alkoxy, acetate, hydroxyl, carbocyclic, heterocyclic or combinations thereof); and X and Y being selected from the group comprising: hydroxyl, acetate, amine, amide, sulfonate, carboxylate, heterocyclic or combinations thereof.

One embodiment of the instant invention describes a method for improving the curl retention of hair comprising the steps of providing a hair care product comprising water, a chelating agent, a copolymer, a pH adjusting agent and a hair treatment composition including a polyvinyl alcohol, polyvinyl alcohol copolymer, vinyl acetate emulsion, acrylic emulsion, vinyl acrylic emulsion, vinyl acetate copolymer emulsion, and, or combinations thereof; applying the hair care product to a portion or all of an individual's hair followed by curling a portion or all of the individual's hair.

The hair care product of the above described method may be a hair spray, a hair gel, a hair mousse or a combination thereof. The polyvinyl alcohol in the above described method may have a molecular weight in the range of 1,000 to 1,000,000 and a concentration in the range of 0.1 to 100 grams per liter (g/L).

In another embodiment the above described method may further comprise a thickener, a preservative, a biocide, a surfactant and, or a combination thereof (see previous descriptions). In still another embodiment, the hair care product of the above described method is water soluble. In yet another embodiment, the method for improving the curl retention of hair wherein the hair treatment composition further comprises a partially hydrolyzed polyvinyl alcohol, a fully hydrolyzed polyvinyl alcohol, or a combination thereof.

In still another embodiment, the method for improving the curl retention of hair of as described above wherein the polyvinyl alcohol copolymer having the following molecular structure:

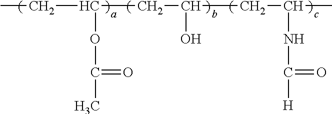

wherein:
a=from about 0 to about 15 mole %;
b=about 0.1 to about 99 mole %;
c=about 0.1 to about 99 mole %; and
   wherein the polyvinyl alcohol copolymer has a molecular weight in the range of 1,000 to 1,000,000.

In still another embodiment, the method for improving the curl retention of hair of as described above wherein the polyvinyl alcohol copolymer having the following molecular structure:

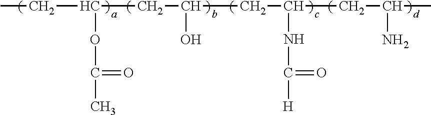

wherein:
a=from about 0 to about 15 mole %;
b=from about 0.1 to about 99 mole %;
c=from about 0.1 to about 99 mole %;
d=from about 0.1 to about 99 mole %; and
   wherein the polyvinyl alcohol copolymer has a molecular weight in the range of 1,000 to 1,000,000

In yet another embodiment, the method for improving the curl retention of hair of as described above wherein the polyvinyl alcohol copolymer emulsion is comprised of a polyvinyl alcohol copolymer with a vinyl emulsion. The vinyl emulsion may be selected from the group comprising: a surfactant stabilized vinyl acrylic emulsion, a vinyl acetate emulsion, a vinyl acetate ethylene emulsion, or combinations thereof.

In one embodiment, for instance, the present disclosure is directed to a skin care product or hair care product that comprises a polyvinyl alcohol copolymer blended with one or more additives. The one or more additives blended with the copolymer may comprise any suitable stability or performance additive. For instance, the stability or performance additive may comprise a polyvinyl pyrrolidone, a poly(vinyl acetate-co-N-vinyl-2-pyrrolidone) copolymer, vinyl caprolactum, a polyvinyl fluoride polymer, a styrene maleic anhydride copolymer, an acrylic polymer, a hydrophobically modified polyvinyl alcohol, a quaternary amine, a polyquaternium, an amine scavenging agent, a cationic surfactant, an amphoteric surfactant, or mixtures thereof.

The above additives when blended with the polyvinyl alcohol copolymer can provide various advantages and benefits. For example, in one embodiment, an additive may be selected that improves the adhesion properties of the polyvinyl alcohol copolymer. When used in a hair treatment composition, for instance, the additive may work in conjunction with the polyvinyl alcohol copolymer to improve shine/gloss, hold, manageability, and curl retention. When used in a personal care composition, on the other hand, the additive may serve to improve adhesion to the skin, improve water resistance, improve lubricity and feel, and/or deliver ultraviolet protection.

The additive can also be used in conjunction with the polyvinyl alcohol copolymer in order to improve storage stability. For instance, the additive may inhibit the N-vinyl formamide from interacting with the other ingredients in the composition that may adversely affect the clarity or haze of the composition.

When contained in a skin moisturizer, for instance, the copolymer described above may be present in an amount from about 0.01% to about 50.0% by weight. The skin moisturizer may contain any suitable moisturizing agent, such as a humectant or emollient. Examples of ingredients that may be contained in the skin moisturizer include, for instance, lactic acid and other hydroxy acids and their salts, glycerin, propylene glycols, butylene glycols, sodium PCA, polyethylene glycols, methoxypolyethylene glycols and the like. Suitable emollients for use in the formulations of the present disclosure include, but are not limited to, PPG-15 stearyl ether, lanolin alcohol, lanolin, lanolin derivatives, cholesterol, petrolatum, isostearyl neopentanoate, octyl stearate, mineral oil, isocetyl stearate, myristyl myristate, octyl dodecanol, dimethicone, phenyl trimethicone, cyclomethicone, dimethiconol, and propylene glycol dicaprylate/dicaprate. Still other ingredients that may be included in the skin moisturizer include urocanic acid, N-acetyl ethanol amine, aloe vera gel, chitosan and its derivatives, glycerides, imidazolidinone, fructose, glucamine, glucose, glucose glutamate, glucuronic acid, honey, hydrogenated starch hydrolysates, glycereths, methylgluceth, urea, xylitol, pyrrolidone carboxylic acids, silicone glycol, ethoxylated glycerin, and the like.

The skin care products may also contain an emulsifier. The emulsifier interacts with the water and oil components to form an emulsion. Suitable emulsifiers for use in the compositions of the present disclosure include, but are not limited to, behentrimonium methosulfate, cetearyl alcohol, non-ionic emulsifiers like polyoxyethylene oleyl ether, PEG-40 stearate, ceteareth-12, ceteareth-20, ceteareth-30, glyceryl stearate, PEG-100 stearate, steareth-2 and steareth-20, or combinations/mixtures thereof, as well as cationic emulsifiers like stearamidopropyl dimethylamine and behentrimonium methosulfate, or combinations/mixtures thereof.

Additional ingredients that may be contained in the skin care products include other water soluble polymers, cationic polymers, antioxidants, preservatives, fragrances and the like, as described herein. Other ingredients include antioxidants. An antioxidant is a natural or synthetic substance added to the formulation to protect it from decay or deterioration due to the reaction with oxygen in the air. Antioxidants prevent deterioration which may lead to the generation of rancidity and non-enzymatic browning reaction products. Suitable antioxidants include, for example, propyl, octyl and dodecyl esters of gallic acid, butylated hydroxyanisole, butylated hydroxytoluene, nordihydroguaiaretic acid, vitamin E, vitamin E acetate, vitamin C, lipoic acid, ubiquinone, Apple Green Tea, Arnica Special, Avocado GW, Bell Pepper Fruit, Black Currant B, Black Currant Green Tea, Blueberry Fruit, Cabbage Rose Extract, Camellia sinensis, Canadian Willowherb, Carrot Root, Camellia Oleifera Extract, Common thyme, Cranberry Green Tea, Echinacea Dry Aqueous Extract, Fennel Fruit, Ginkgo Biloba, Glycine Max (soybean seed), Goldenseal, Grapefruit, Grape Seed Extract and constituents thereof (proanthocyanidins), Grapefruit Green Tea, Green Tea, catechin constituents of Green Tea that include epigallocatechin gallate, epicathechin gallate, Green Tea Extra, Green Tea HS, Lime Blossom, Orange Green Tea, Rosemary Plant, Sea Parsley, St. John's Wort W/S, Strawberry Fruit, Tomato Root, Turkish Oregano, Wheat Seed, White Mistle Toe, White Tea, Yarrow, Yucca 70, Yucca Extract Powder, and combinations thereof.

Fragrances can also be added to the skin care products described herein. Fragrances are aromatic compounds which can impart an aesthetically pleasing aroma to the composition. Typical fragrances include, for example, aromatic materials extracted from botanical sources such as rose petals, gardenia blossoms, and jasmine flowers which can be used alone or in any combination to create essential oils. Additionally, alcoholic extracts may be prepared for fragrances.

In addition to skin moisturizers, the copolymer of the present disclosure is also well suited for use in sunscreen and sun protection compositions. The copolymer may be present in the sunscreen composition in an amount from about 0.01% to about 75% by weight. As described above, the polyvinyl alcohol and N-vinyl formamide copolymer improves skin adhesion thus increasing the longevity of the sunscreen composition. Any sunscreen agent and sun protecting agent known in the art or apparent to the skilled artisan may be used in the sunscreen and sun protection composition.

Specific suitable sunscreen agents include, for example: p-aminobenzoic acid, its salts and its derivatives (ethyl, isobutyl, glyceryl esters; p-dimethylaminobenzoic acid); Anthranilates (i.e., o-aminobenzoates; methyl, menthyl, phenyl, benzyl, phenylethyl, linalyl, terpinyl, and cyclohexenyl esters); Salicylates (amyl, phenyl, benzyl, menthyl, glyceryl, and dipropylene glycol esters); Cinnamic acid derivatives (methyl and benzyl esters, alpha-phenyl cinnamonitrile; butyl cinnamoyl pyruvate); Dihydroxycinnamic acid derivatives (umbelliferone, methylumbelliferone, methylaceto-umbelliferone); Trihydroxycinnamic acid derivatives (esculetin, methylesculetin, daphnetin, and the glucosides, esculin and daphnin); Hydrocarbons (diphenylbutadiene, stilbene); Dibenzalacetone and benzalacetophenone; Naphtholsulfonates (sodium salts of 2-naphthol-3,3-disulfonic and of 2-naphthol-6,8-disulfonic acids); Dihydroxynaphthoic acid and its salts; o- and p-Hydroxybiphenyldisulfonates; Coumarin derivatives (7-hydroxy, 7-methyl, 3-phenyl); Diazoles (2-acetyl-3-bromoindazole, phenyl benzoxazole, methyl naphthoxalole, various aryl benzothiazoles); Quinine salts (bisulfate, sulfate, chloride, oleate, and tannate); quinoline derivatives (8-hydroxyquinoline salts, 2-phenylquinoline); Hydroxy- or methoxy substituted benzophenones; Uric and vilouric acids; Tannnic acid and its derivatives (e.g., hexaethylether); (Butyl carbityl) (6-propyl piperonyl)ether; Hydroquinone; Benzophenones (Oxybenzene, Sulisobenzone, Dioxybenzone, Benzoresorcinol, 2,2′,4,4′-Tetrahydroxybenzophenone, 2,2′-Dihydroxy-4,4′-dimethoxybenzophenone, Octabenzone; 4-Isopropyhldibenzoylmethane; Butylmethoxydibenzoylmethane; Etocrylene; and 4-isopropyl-di-benzoylmethane; titanium dioxide, iron oxide, zinc oxide, and mixtures thereof. Other cosmetically-acceptable sunscreen agents and concentrations (percent by weight of the total cosmetic sunscreen composition) include diethanolamine methoxycinnamate (10% or less), ethyl-4-[bis(hydroxypropyl)]aminobenzoate (5% or less), glyceryl aminobenzoate (3% or less), 4-isopropyl dibenzoylmethane (5% or less), 4-methylbenzylidene camphor (6% or less), terephthalylidene dicamphor sulfonic acid (10% or less), and sulisobenzone (also called benzophenone-4, 10% or less).

Sunscreen compositions made in accordance with the present disclosure may also include any of the ingredients listed above with respect to skin moisturizers.

In one embodiment of the present invention, a skin care product comprises a skin moisturizer or a sunscreen/sun protecting agent and a skin treatment composition including a polyvinyl alcohol polymer, wherein the polyvinyl alcohol copolymer has the following molecular structure:

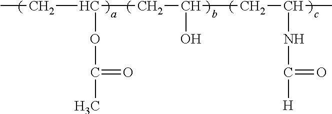

wherein:
a=from about 0 to about 15 mole %;
b=about 5 to about 99 mole %; and
c=about 1 to about 99 mole %.

In another embodiment of the present invention, a skin care product comprises a skin moisturizer or a sunscreen/sun protecting agent and a skin treatment composition including a polyvinyl alcohol polymer, wherein the polyvinyl alcohol copolymer has the following molecular structure:

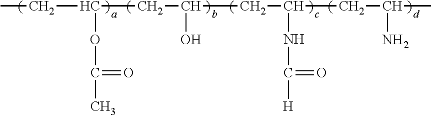

wherein:
a=from about 0 to about 15 mole %;
b=from about 5 to about 99 mole %;
c=from about 5 to about 95 mole %;
d=from about 1 to about 95 mole %.

In still another embodiment of the present invention, a skin care product comprising a skin moisturizer or a sunscreen/sun protecting agent and a skin treatment composition, wherein the skin care composition acts as a chelating agent, a skin conditioning agent, a preservative, a salt tolerance agent, a gellant, a film forming agent, a firming agent, a skin protectant, a shine/gloss enhancer, a toning agent, a moisturizer, a emollient, a anti-wrinkle agent, a water repellent enhancer, a carrier and/or lubricant or a combination thereof.

In yet another embodiment of the present invention, a skin care product or hair care product comprising a polyvinyl alcohol copolymer; and/or a stability or performance additive comprising a polyvinyl pyrrolidone, a poly(vinyl acetate-co-N-vinyl-2-pyrrolidone) copolymer, vinyl caprolactum, a polyvinyl fluoride polymer, a styrene maleic anhydride copolymer, an acrylic polymer, a hydrophobically modified polyvinyl alcohol, a quaternary amine, a polyquaternium, an amine scavenging agent, a cationic surfactant, an amphoteric surfactant, or mixtures thereof.

In one embodiment, a skin care product or hair care product as defined in the previous paragraph, wherein the polyvinyl alcohol copolymer has the following molecular structure:

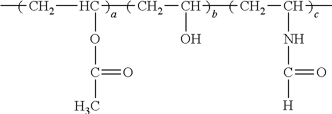

wherein:
a=from about 0 to about 15 mole %;
b=about 5 to about 99 mole %; and
c=about 1 to about 95 mole %.

In another embodiment, a skin care product or hair care product as defined above, wherein the polyvinyl alcohol copolymer has the following molecular structure:

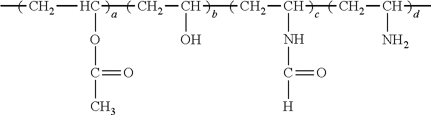

wherein:
a=from about 0 to about 15 mole %;
b=from about 5 to about 99 mole %;
c=from about 5 to about 95 mole %;
d=from about 1 to about 95 mole %.

In still another embodiment, a skin care product or hair care product as defined above, wherein the product further comprises a chelating agent, a preservative, and a pH adjuster.

In yet another embodiment, a skin care product or hair care product as defined above, wherein the product being a hair spray or hair gel for curl retention, the hair care product having a curl retention of at least about 70% at 90% relative humidity for 0.75 hours.

In another embodiment, a skin care product or hair care product as defined above, wherein the product being a sunscreen lotion, the product further comprising a sunscreen agent, the sunscreen agent comprising the zinc oxide, titanium dioxide, iron oxide, a para-amino benzoate, a salicylate, a cinnamate, a benzophenone, a menthyl anthralinate, a digalloyl trilleate, or mixtures thereof.

In still another embodiment, a skin care product or hair care product as defined above, wherein the product being a skin moisturizer, and wherein the product further comprises glycerine, a dimethicone or a derivative thereof, a propylene glycol, a lanolin or a derivative thereof, a petrolatum, or mixtures thereof.

In yet another embodiment, a skin care product or hair care product as defined above, wherein the copolymer being used as an additive and wherein the product further comprises rheology modifiers, hair dyes, propellants, biocides, thickeners, viscosity enhancing agents, surfactants, pigments, dispersions aids, moisture retention agents, waxes, oils, pigments, encapsulating agents, tanning aids, vitamins, active ingredients, proteins, preservatives, plasticizers, film formers, hydrating substances, silicones, UV filters and absorbers, skin whiteners, alpha hydroxyacids, antioxidants, essential oils, anti aging products, emollients, emulsifiers and emulsion stabilizers, mixing aide, suspension agents, emulsifying agents, sensory modifiers, humectants, slip agents, solvency, gelling agents, fragrance fixatives, foam enhancers, deposition agents, water proofing agents, hair protectants, opacifiers, pearlizers, plant extracts, polyquaterniums, various silicone derivatives, terpenes hydrocarbons, hydrocarbons or other oil components, antiseptic, various amino acids, activators, flake cutting, pigment or other general known additive thereof, anti-seborrheic agents, diluents, fragrance, colorants, derivatives thereof, or mixtures thereof.

26. A skin care product as defined in claim 25, wherein the acrylate polymer comprises a crosspolymer.

27. A skin care product as defined in claim 22, wherein the polyvinyl alcohol copolymer has the following molecular structure:

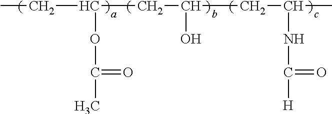

wherein:
a=from about 0 to about 15 mole %;
b=about 5 to about 99 mole %; and
c=about 1 to about 99 mole %.

28. A skin care product as defined in claim 22, wherein the polyvinyl alcohol copolymer has the following molecular structure:

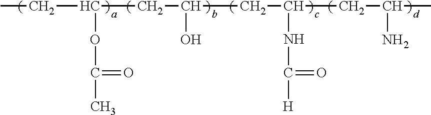

wherein:
a=from about 0 to about 15 mole %;
b=from about 5 to about 99 mole %;
c=from about 5 to about 95 mole %;
d=from about 1 to about 95 mole %.

29. A skin care product of claim 22 wherein the skin treatment composition acts as a chelating agent, a skin conditioning agent, a preservative, a salt tolerance agent, a gellant, a film forming agent, a firming agent, a skin protectant, a shine/gloss enhancer, a toning agent, a moisturizer, a emollient, a anti-wrinkle agent, a water repellent enhancer, a carrier and/or lubricant or a combination thereof.

30. A skin care product or hair care product comprising:
a polyvinyl alcohol copolymer; and/or
a stability or performance additive comprising a polyvinyl pyrrolidone, a poly(vinyl acetate-co-N-vinyl-2-pyrrolidone) copolymer, vinyl caprolactum, a polyvinyl fluoride polymer, a styrene maleic anhydride copolymer, an acrylic polymer, a hydrophobically modified polyvinyl alcohol, a quaternary amine, a polyquaternium, an amine scavenging agent, a cationic surfactant, an amphoteric surfactant, or mixtures thereof.

31. A skin care product or hair care product as defined in claim 30, wherein the stability or performance additive comprises the polyvinyl pyrrolidone.

32. A skin care product or hair care product as defined in claim 30, wherein the stability or performance additive comprises the poly(vinyl acetate-co-N-vinyl-2-pyrrolidone) copolymer.

33. A skin care product or hair care product as defined in claim 30, wherein the stability or performance additive comprises the vinyl caprolactum.

34. A skin care product or hair care product as defined in claim 30, wherein the stability or performance additive comprises the polyvinyl fluoride polymer.

35. A skin care product or hair care product as defined in claim 30, wherein the stability or performance additive comprises the styrene maleic anhydride copolymer.

36. A skin care product or hair care product as defined in claim 30, wherein the stability or performance additive comprises the acrylic polymer.

37. A skin care product or hair care product as defined in claim 30, wherein the stability or performance additive comprises the hydrophobically modified polyvinyl alcohol.

38. A skin care product or hair care product as defined in claim 30, wherein the polyvinyl alcohol copolymer has the following molecular structure:

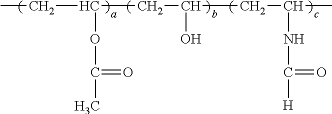

wherein:
a=from about 0 to about 15 mole %;
b=about 5 to about 99 mole %; and
c=about 1 to about 95 mole %.

39. A skin care product or hair care product as defined in claim 30, wherein the polyvinyl alcohol copolymer has the following molecular structure:

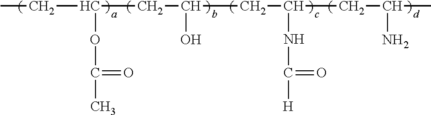

wherein:
a=from about 0 to about 15 mole %;
b=from about 5 to about 99 mole %;
c=from about 5 to about 95 mole %;
d=from about 1 to about 95 mole %.

40. A skin care product or hair care product as defined in claim 30, further comprising a thickener, the thickener comprising an acrylate polymer.

41. A skin care product or hair care product as defined in claim 30, wherein the stability or performance additive comprises the quaternary amine.

42. A skin care product or hair care product as defined in claim 30, wherein the stability or performance additive comprises the polyquaternium.

43. A skin care product or hair care product as defined in claim 30, wherein the stability or performance additive comprises the amine scavenging agent, and wherein the amine scavenging agent comprises a guanadine.

44. A skin care product or hair care product as defined in claim 30, wherein the stability or performance additive comprises the cationic surfactant.

45. A skin care product or hair care product as defined in claim 30, wherein the stability or performance additive comprises the amphoteric surfactant.

46. A skin care product or hair care product as defined in claim 30, wherein the product further comprises a chelating agent, a preservative, and a pH adjuster.

47. A skin care product or hair care product as defined in claim 30, wherein the product being a hair spray or hair gel for curl retention, the hair care product having a curl retention of at least about 70% at 90% relative humidity for 0.75 hours.

48. A skin care product or hair care product as defined in claim 30, wherein the product being a sunscreen lotion, the product further comprising a sunscreen agent, the sunscreen agent comprising the zinc oxide, titanium dioxide, iron oxide, a para-amino benzoate, a salicylate, a cinnamate, a benzophenone, a menthyl anthralinate, a digalloyl trilleate, or mixtures thereof.

49. A skin care product or hair care product as defined in claim 30, wherein the product being a skin moisturizer, and wherein the product further comprises glycerine, a dimethicone or a derivative thereof, a propylene glycol, a lanolin or a derivative thereof, a petrolatum, or mixtures thereof.

50. A skin care product or hair care product as defined in claim 30, wherein the copolymer being used as an additive and wherein the product further comprises rheology modifiers, hair dyes, propellants, biocides, thickeners, viscosity enhancing agents, surfactants, pigments, dispersions aids, moisture retention agents, waxes, oils, pigments, encapsulating agents, tanning aids, vitamins, active ingredients, proteins, preservatives, plasticizers, film formers, hydrating substances, silicones, UV filters and absorbers, skin whiteners, alpha hydroxyacids, antioxidants, essential oils, anti aging products, emollients, emulsifiers and emulsion stabilizers, mixing aide, suspension agents, emulsifying agents, sensory modifiers, humectants, slip agents, solvency, gelling agents, fragrance fixatives, foam enhancers, deposition agents, water proofing agents, hair protectants, opacifiers, pearlizers, plant extracts, polyquaterniums, various silicone derivatives, terpenes hydrocarbons, hydrocarbons or other oil components, antiseptic, various amino acids, activators, flake cutting, pigment or other general known additive thereof, anti-seborrheic agents, diluents, fragrance, colorants, derivatives thereof, or mixtures thereof.

### EXAMPLES

The following examples further illustrate the instant invention. The hair care formulations set forth in Table 1 were applied to hair tresses which were 1 inch by 7 inches using a 1 milliliter syringe. Thereafter the treated tresses were subjected to testing according to the test outlined below using a Nor-Lake Scientific Temperature Test Chamber (Instruction Manual 5/08 132574 Rev. C was utilized). The results of these experimental trials are set forth in Table 1.

Humidity Resistance—Percent Curl Retention. The hair setting efficacy of a composition can be evaluated by the retention of the hair set at high humidity of about 90% Relative Humidity (RH). The ability to hold a curl set on hair after absorption of water from the applied composition and from the surrounding atmosphere can be measured employing the well known techniques commonly referred to as high humidity curl retention (HHCR). Descriptions of the HHCR methodology are readily found in the cosmetic literature. See, for example, Ch. 30, Harry's Cosmeticology, 8th Ed., M. J. Rieger, Ph.D. (ed.), 666 667, Chemical Publishing Co., Inc., New York, N.Y. (2000), and Diaz et al., J. Soc. Cosmet. Chem., 34, 205 212 (July 1983), the relevant disclosures of each are incorporated herein by reference.

Tresses of commercially blended Caucasian, untreated (virgin) human hair are prepared employing natural brown or black color European hair supplied by International Hair Importers and Products Inc., New York. Each hair tress (about 3 grams weight) is about 7 inches (about 18 cm) in length and is securely anchored with glue at the scalp (root) end portion. Prior to use, each hair tress is pre-cleaned by washing with a dilute aqueous solution of sodium lauryl sulfate (10% SLS), rinsing thoroughly with deionized water at ambient room temperature and drying with towel blotting. The initial extended length of the hair (Lₑ) is measured. About 0.8 grams of polymer-containing composition to be evaluated are applied to the hair tress and distributed from the scalp to end portion. The treated hair tress is then wrapped around a hair curler having an outer diameter of about 3 cm, and allowed to dry on the curler overnight at an ambient room temperature of about 21 to 23° C. After drying, the curler is carefully removed, leaving the hair styled into a single curl, the initial length of the hair curl (Lᵢ) is measured, and the curled hair tress is vertically hung in a humidity chamber set at an ambient temperature of about 26 to 27° C. and ambient high humidity of about 90% RH.

High humidity curl retention (HHCR) is determined by measuring the length of the hair curl as the curl relaxed after selected intervals (Lₜ) of exposure to humidity. The following equation is used to calculate percent curl retention, relative to the initial curl length (Lᵢ) and initial extended length of the hair (Lₑ).

$$\% Curl Retention = \frac{{L}_{e} - {L}_{t}}{{L}_{e} - {L}_{i}} \times 100 \%$$

The change in curl length (droop, helix formation) is periodically measured and visually observed. Measurements are taken initially, and at 0.25 hour intervals over a total exposure period of about 1.25 to 1.75 hours, and then at hourly intervals thereafter over a total exposure period of up to about 8 hours, with a final reading being taken after a total exposure period of about 24 hours. HHCR efficacy of about 70% or more curl retention (CR) for a minimum period of at least about 0.75 hours at about 90% RH is referred to herein as “T@70% CR”, and is a conventional benchmark for good high humidity resistance and is referred to herein as good hair setting efficacy. HHCR efficacy reported herein for a selected time interval is identified as “% CR @T-x”, where x is the hourly interval or fraction thereof. Hair setting efficacy of 70% CR @T-1.25 hours or longer is judged very good, and 70% CR @T-3 hours or longer is judged as excellent. Hair setting efficacy is judged weak when % curl retention was not more than about 50%.

TABLE 1
Humidity Chamber Test on Virgin Hair - 32 degrees Celsius, 90% Humidity
Gel Loading 0.50 ml on Hair

| | | | Test Hours | | | | | | | | |
|---|---|---|---|---|---|---|---|---|---|---|---|
| | | Sample | | | 2 Hr Curl | | 4 HrCurl | | 8 Hr Curl | | 24 Hr Curl |
| Formula | Contents | # | 0 | 2 | Retention | 4 | Retention | 8 | Retention | 24 | Retention |
| 1 | Water, Chelating | 1a | 2.9 | 3.0 | 99.3 | 3 | 99.3 | 3.0 | 99.3 | 3.0 | 99.3 |
| | Agent, Copolymer | 1b | 2.6 | 3.0 | 97.4 | 3 | 97.4 | 3.0 | 97.4 | 3.0 | 97.4 |
| | pH adjuster (NaOH, | | | | | | | | | | |
| | benzophenone 4) | | | | | | | | | | |
| | Preservative | | | | | | | | | | |
| | 6% N-vinyl formamide/ | | | | | | | | | | |
| | 94% PVOH | | | | | | | | | | |
| 2 | Water, Chelating | 2a | 3.0 | 3.0 | 100.0 | 3.3 | 98.0 | 3.3 | 98.0 | 3.5 | 96.6 |
| | Agent, Copolymer | 2b | 2.8 | 3.1 | 98.0 | 3.3 | 96.7 | 3.3 | 96.7 | 3.3 | 96.7 |
| | pH adjuster (NaOH, | | | | | | | | | | |
| | benzophenone 4, 2 | | | | | | | | | | |
| | amino-2-methyl-1-pro- | | | | | | | | | | |
| | panol) | | | | | | | | | | |
| | Preservative | | | | | | | | | | |
| | 6% N-vinyl formamide/ | | | | | | | | | | |
| | 94% PVOH | | | | | | | | | | |
| 3 | Water, Chelating | 3a | 2.7 | 2.7 | 100.0 | 2.7 | 100.0 | 2.7 | 100.0 | 2.7 | 100.0 |
| | Agent, Copolymer | 3b | 2.8 | 3.3 | 96.7 | 3.3 | 96.7 | 3.5 | 95.3 | 3.6 | 94.7 |
| | pH adjuster (2 | | | | | | | | | | |
| | amino-2-methyl-1-pro- | | | | | | | | | | |
| | panol) | | | | | | | | | | |
| | Biocide | | | | | | | | | | |
| | 6% N-vinyl formamide/ | | | | | | | | | | |
| | 94% PVOH | | | | | | | | | | |
| 3(2) | Water, Chelating | 3(2)a | 3.0 | 3.0 | 100.0 | 3.0 | 100.0 | 3.0 | 100.0 | 3.0 | 100.0 |
| | Agent, Copolymer | 3(2)b | 2.8 | 3.0 | 98.7 | 3.0 | 98.7 | 3.1 | 98.0 | 3.1 | 98.0 |
| | pH adjuster (2 | | | | | | | | | | |
| | amino-2-methyl-1-pro- | | | | | | | | | | |
| | panol) | | | | | | | | | | |
| | Preservative | | | | | | | | | | |
| | 6% N-vinyl formamide/ | | | | | | | | | | |
| | 94% PVOH | | | | | | | | | | |
| 4 | Water, Chelating | 4a | 2.6 | 2.9 | 98.0 | 3.0 | 97.4 | 3.0 | 97.4 | 3.0 | 97.4 |
| | Agent, Copolymer | 4b | 2.7 | 3.0 | 98.0 | 3.0 | 98.0 | 3.1 | 97.3 | 3.5 | 94.7 |
| | pH adjuster (2 | | | | | | | | | | |
| | amino-2-methyl-1-pro- | | | | | | | | | | |
| | panol, triethanolamine) | | | | | | | | | | |
| | Biocide | | | | | | | | | | |
| | 6% N-vinyl formamide/ | | | | | | | | | | |
| | 94% PVOH | | | | | | | | | | |
| 6 | Water, Chelating | 6a | 2.9 | 3.2 | 98.0 | 3.5 | 96.0 | 4.0 | 92.6 | 9.5 | 55.6 |
| | Agent, Copolymer | 6b | 2.5 | 3.2 | 95.4 | 3.5 | 93.4 | 4.5 | 86.9 | 9.5 | 54.1 |
| | pH adjuster (2 | | | | | | | | | | |
| | amino-2-methyl-1-pro- | | | | | | | | | | |
| | panol) | | | | | | | | | | |
| | Biocide | | | | | | | | | | |
| | PVOH-Hydrolyzed | | | | | | | | | | |
| | 6% N-vinyl formamide/ | | | | | | | | | | |
| | 94% PVOH | | | | | | | | | | |
| 7 | Water, Chelating | 7a | 2.5 | 3.2 | 95.4 | 3.2 | 95.4 | 3.2 | 95.4 | 3.2 | 95.4 |
| | Agent, Copolymer | 7b | 2.5 | 2.8 | 98.0 | 2.8 | 98.0 | 3 | 96.7 | 3.5 | 93.4 |
| | pH adjuster (2 | | | | | | | | | | |
| | amino-2-methyl-1-pro- | | | | | | | | | | |
| | panol, triethanolamine) | | | | | | | | | | |
| | Biocide | | | | | | | | | | |
| | PVOH-Partially | | | | | | | | | | |
| | Hydrolyzed | | | | | | | | | | |
| | 6% N-vinyl formamide/ | | | | | | | | | | |
| | 94% PVOH | | | | | | | | | | |
| 8 | Water, Chelating | 8a | 2.5 | 4 | 90.2 | 4.5 | 86.9 | 5.2 | 82.3 | 5.4 | 81.0 |
| | Agent, Copolymer | 8b | 2.8 | 3.7 | 94.0 | 4.0 | 92.0 | 4.0 | 92.0 | 4.5 | 88.6 |
| | pH adjuster (NaOH, 2 | | | | | | | | | | |
| | amino-2-methyl-1-pro- | | | | | | | | | | |
| | panol) | | | | | | | | | | |
| | Biocide | | | | | | | | | | |
| | PVOH-Partially | | | | | | | | | | |
| | Hydrolyzed | | | | | | | | | | |
| | 6% N-vinyl formamide/ | | | | | | | | | | |
| | 94% PVOH | | | | | | | | | | |
| 9 | Water, Chelating | 9a | 2.5 | 16 | 8.3 | 17 | 5.0 | 17 | 3.7 | 17 | 3.7 |
| | Agent, Copolymer | 9b | 2.5 | 15 | 18.1 | 15 | 14.8 | 16 | 11.6 | 16 | 11.6 |
| | Thickener | | | | | | | | | | |
| | Biocide | | | | | | | | | | |
| | 6% N-vinyl formamide/ | | | | | | | | | | |
| | 94% PVOH | | | | | | | | | | |
| 11 | Water, Chelating | 11a | 2.5 | 2.9 | 97.4 | 3.0 | 96.7 | 3.0 | 96.7 | 3.0 | 96.7 |
| | Agent, Copolymer | 11b | 3.0 | 3.5 | 96.6 | 3.5 | 96.6 | 3.5 | 96.6 | 3.5 | 96.6 |
| | Thickener | | | | | | | | | | |
| | pH adjuster | | | | | | | | | | |
| | (benzophenone 4, 2 | | | | | | | | | | |
| | amino-2-methyl-1-pro- | | | | | | | | | | |
| | panol) | | | | | | | | | | |
| | Preservative | | | | | | | | | | |
| | 6% N-vinyl formamide/ | | | | | | | | | | |
| | 94% PVOH | | | | | | | | | | |
| 12 | Water, Chelating | 12a | 2.6 | 2.6 | 100.0 | 2.6 | 100.0 | 2.6 | 100.0 | 2.6 | 100.0 |
| | Agent, Copolymer | 12b | 2.5 | 3.0 | 96.7 | 3.2 | 95.4 | 3.3 | 94.8 | 3.8 | 91.5 |
| | Thickener | | | | | | | | | | |
| | pH adjuster (NaOH, | | | | | | | | | | |
| | benzophenone 4, 2 | | | | | | | | | | |
| | amino-2-methyl-1-pro- | | | | | | | | | | |
| | panol) | | | | | | | | | | |
| | Preservative | | | | | | | | | | |
| | 6% N-vinyl formamide/ | | | | | | | | | | |
| | 94% PVOH | | | | | | | | | | |
| 13 | Water, Chelating | 13a | 2.2 | 2.8 | 96.1 | 3.0 | 94.9 | 3.0 | 94.9 | 3.0 | 94.9 |
| | Agent, Copolymer | 13b | 2.0 | 2.5 | 96.8 | 2.8 | 94.9 | 2.8 | 94.9 | 3.0 | 93.7 |
| | pH adjuster | | | | | | | | | | |
| | (triethanolamine) | | | | | | | | | | |
| | Biocide | | | | | | | | | | |
| | 6% N-vinyl formamide/ | | | | | | | | | | |
| | 94% PVOH | | | | | | | | | | |

$Curl Retention = \frac{Length of Hair ( {7}^{″} ) - Length of Hair after chamber}{Length of Hair ( {7}^{″} ) - Length of Hair before chamber ( initial curl )} \times 100$

Table 1 contains data which illustrates the effectiveness of embodiments of the instant invention in curl retention of hair at 32 degrees Celsius and 90 percent relative humidity.

What is claimed is:

1. A hair care product comprising:
water;
a chelating agent;
a copolymer;
a pH adjusting agent; and
a hair treatment composition including a polyvinyl alcohol, polyvinyl alcohol copolymer, vinyl acetate emulsion, acrylic emulsion, vinyl acrylic emulsion, vinyl acetate copolymer emulsion, and, or combinations thereof.

2. The hair care product of claim 1 wherein said polyvinyl alcohol having a molecular weight in the range of 1,000 to 1,000,000 and having a concentration in the range of 0.1 to 100 grams per liter (g/L).

3. The hair care product of claim 1 wherein the hair treatment composition further comprising a thickener, a preservative, a biocide, a surfactant and, or a combination thereof.

4. The hair care product of claim 1 wherein the hair care product being water soluble.

5. The hair care product of claim 1 wherein the hair treatment composition further comprising a partially hydrolyzed polyvinyl alcohol, a fully hydrolyzed polyvinyl alcohol, or a combination thereof.

6. The hair care product of claim 1, wherein the polyvinyl alcohol copolymer having the following molecular structure: 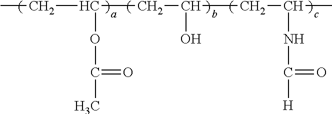
wherein:
a=from about 0 to about 15 mole %;
b=about 0.1 to about 99 mole %;
c=about 0.1 to about 99 mole %; and
wherein said polyvinyl alcohol copolymer having a molecular weight in the range of 1,000 to 1,000,000.

7. The hair care product of claim 1, wherein the polyvinyl alcohol copolymer having the following molecular structure: 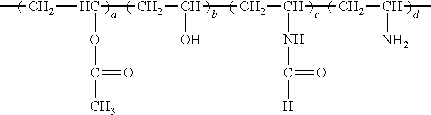
wherein:
a=from about 0 to about 15 mole %;
b=from about 0.1 to about 99 mole %;
c=from about 0.1 to about 99 mole %;
d=from about 0.1 to about 99 mole %; and
wherein said polyvinyl alcohol copolymer having a molecular weight in the range of 1,000 to 1,000,000.

8. The hair care product of claim 1 wherein said hair treatment composition being comprised of a polyvinyl alcohol, a polyvinyl alcohol copolymer with a vinyl acetate, a vinyl acetate copolymer, an acrylic emulsion or a vinyl acrylic emulsion.

9. The hair care product of claim 8 wherein said emulsion being selected from the group comprising: vinyl acrylic emulsion, a vinyl acetate emulsion, a vinyl acetate ethylene emulsion, an acrylic emulsion and, or combinations thereof.

10. The hair care product of claim 1 wherein said hair care product being a hair spray, a hair gel, a hair mousse or a combination thereof.

11. The hair care product of claim 1 wherein said hair treatment composition acts as a chelating agent, a conditioning agent, a preservative, a curl retention agent, a salt tolerance agent, a gellant, a film forming agent, a holding agent, a flake reducer, a relaxer enhancing agent, a fortifying agent, a shine/gloss enhancer, a strengthener, a humidity resistance agent, a carrier and/or lubricant or a combination thereof.

12. A method for improving the curl retention of hair comprising the steps of:
providing a hair care product comprising:
water;
a chelating agent;
a copolymer;
a pH adjusting agent; and
a hair treatment composition including a polyvinyl alcohol, polyvinyl alcohol copolymer, vinyl acetate emulsion, acrylic emulsion, vinyl acrylic emulsion, vinyl acetate copolymer emulsion, and, or combinations thereof;
applying said hair care product to a portion or all of an individual's hair;
curling a portion or all of said individual's hair.

13. The method for improving the curl retention of hair of claim 12 wherein said polyvinyl alcohol having a molecular weight in the range of 1,000 to 1,000,000 and having a concentration in the range of 0.1 to 100 grams per liter (g/L).

14. The method for improving the curl retention of hair of claim 12 wherein the hair treatment composition further comprising a thickener, a preservative, a biocide, a surfactant and, or a combination thereof.

15. The method for improving the curl retention of hair of claim 12 wherein the hair care product being water soluble.

16. The method for improving the curl retention of hair of claim 12 wherein the hair treatment composition further comprising a partially hydrolyzed polyvinyl alcohol, a fully hydrolyzed polyvinyl alcohol, or a combination thereof.

17. The method for improving the curl retention of hair of claim 12 wherein the polyvinyl alcohol copolymer having the following molecular structure: 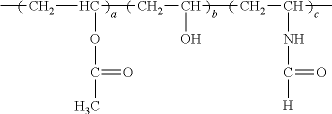
wherein:
a=from about 0 to about 15 mole %;
b=about 0.1 to about 99 mole %;
c=about 0.1 to about 99 mole %; and
wherein said polyvinyl alcohol copolymer having a molecular weight in the range of 1,000 to 1,000,000.

18. The method for improving the curl retention of hair of claim 12 wherein the polyvinyl alcohol copolymer having the following molecular structure: 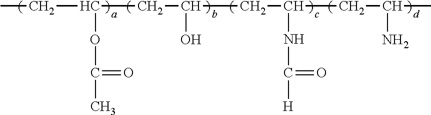
wherein:
a=from about 0 to about 15 mole %;
b=from about 0.1 to about 99 mole %;
c=from about 0.1 to about 99 mole %;
d=from about 0.1 to about 99 mole %; and
wherein said polyvinyl alcohol copolymer having a molecular weight in the range of 1,000 to 1,000,000.

19. The method of claim 12 wherein said polyvinyl alcohol copolymer emulsion being comprised of a polyvinyl alcohol copolymer with a vinyl emulsion.

20. The method of claim 19 wherein said vinyl emulsion being selected from the group comprising: a surfactant stabilized vinyl acrylic emulsion, a vinyl acetate emulsion, a vinyl acetate ethylene emulsion, or combinations thereof.

21. The method of claim 12 wherein said hair care product being a hair spray, a hair gel, a hair mousse or a combination thereof.

22. A skin care product comprising:
a skin treatment composition including a polyvinyl alcohol, polyvinyl alcohol copolymer, vinyl acetate emulsion, acrylic emulsion, vinyl acrylic emulsion, vinyl acetate copolymer emulsion, and, or combinations thereof; and
a skin moisturizer or a sunscreen/sun protecting agent.

23. A skin care product as defined in claim 22, wherein the skin care product contains the sunscreen agent, the sunscreen agent comprising zinc oxide, titanium dioxide, iron oxide, a para-amino benzoate, a salicylate, a cinnamate, a benzophenone, a menthyl anthralinate, a digalloyl trilleate, or mixtures thereof.

24. A skin care product as defined in claim 22, wherein the skin care product contains the skin moisturizer, the skin moisturizer comprising glycerine, a dimethicone or a derivative thereof, propylene glycol, lanolin or a derivative thereof, petrolatum, or mixtures thereof.

25. A skin care product as defined in claim 22, further comprising a thickener, the thickener comprising an acrylate polymer.
